(19) United States
(12) Patent Application Publication
(10) Pub. No.: US 2020/0107778 A1
(43) Pub. Date: Apr. 9, 2020

(54) Using biometrics to measure, catalog, analyze, predict and/or monitor emergency responder stress to optimize performance and health

(71) Applicant: Provicta LLC, Wayzata, MN (US)

(72) Inventors: Mike Goldstein, Excelsior, MN (US); Peter Lecy, Wayzata, MN (US); Stephen Mase, Minneapolis, MN (US)

(21) Appl. No.: 16/593,044

(22) Filed: Oct. 4, 2019

Related U.S. Application Data
(60) Provisional application No. 62/741,824, filed on Oct. 5, 2018.

(51) Int. Cl. A61B 5/00; A61B 5/0205; A61B 5/16

(52) U.S. Cl. CPC A61B 5/4884; A61B 5/0205; A61B 5/165; A61B 5/02405; A61B 5/7275; A61B 5/7278; A61B 5/743; A61B 5/486

(57) ABSTRACT

A platform can include a processor and a memory with instructions that cause the processor to: receive biometric information from a plurality of public safety personnel; analyze the biometric information to calculate a stress index; and use the stress index to define and monitor stress in the public safety personnel.

### RELATED APPLICATION(S)

This patent application claims the benefit of U.S. Patent Application No. 62/741,824 filed on Oct. 5, 2018, the entirety of which is hereby incorporated by reference.

### BACKGROUND

In the emergency responder (e.g., law enforcement) realm, the societal need to optimize individual and group performance especially in critical situations is significant. While a number of helpful, and in some cases, costly lessons have been learned through a complex mix of planned operations, trial and error, and simple happenstance, one critical aspect is clear: nothing can replace the human decision-making process that police officers must effectively employ in their service.

The law enforcement industry has long-sought through training and fitness activities to aid police officers in making better decisions more quickly with precision and confidence, and in this context, cognition plays a major role. Optimizing the performance of police officers is a psychophysiological process with cognition being a critical factor.

Stress is commonly used to describe and quantify the body's response to an emotional experience that is accompanied by predictable biochemical, physiological and behavioral changes. No stress during an incident suggests the individual is not ready or fully prepared. As stress gets too high, the individual will begin to lose cognitive processing capabilities. ‘Resilience’ is the capacity to recover quickly from these emotional events and the ability to effectively cope with a crisis.

### SUMMARY

From both a pre-service and in-service perspective, the platforms described herein will provide emergency responders the opportunity to capture useful physiological metrics that establish baselines to better study how stress-induced events affect an emergency responder's performance during both routine and critical incidents. With that knowledge, more specific advanced conditioning techniques, analogous to personalized medicine, both in the academy and in-service training settings, would be applied to assist in optimizing one's performance and over time, help increase an officer's resiliency to stress. Through this optimization, performance levels could congruently increase, resulting in less liability for all.

For an individual, there is a functional threshold for each class of events that represents the minimum level of cognitive acuity below which an individual is not at a level sufficient to properly engage their cognitive faculties. Correspondingly, there is a maximum threshold above which the individual is over-stimulated and cognitively frozen-out. These thresholds bound within a range of optimal stress levels. When the individual maintains a steady state between the lower and higher thresholds, he or she is in the optimal zone for that event class. Optimum performance equates to 1) establishing the zone and 2) staying within the zone for the duration of an event.

These thresholds surrounding a person's ideal stress index will vary over time and change based on fitness levels, life events and cumulative experiences, for example Post Traumatic Stress Disorder (PTSD). Keeping these longitudinal thresholds current assures both analytical integrity and the ability to maintain optimal performance levels.

Further, with the creation of a longitudinally designed baseline stress index, predictive measures will result through the use of artificial intelligence and machine learning to enhance candidate selection processes for critical jobs and to establish a sound foundation to diagnose changes in monitored behavior and performance. Thereafter, these diagnostic and prescriptive cues can be further interpreted by professionals in medicine, fitness, industry, etc.

Through the appropriate monitoring, law enforcement will have the ability to ensure, to the best of its capabilities, that police officers stay within acceptable margins of vigilance, whereby they will be poised to manage what they encounter in a prudent and appropriate manner. If the margins should shift in one direction or another, a corrective reminder or alert can be provided to the officer. In the case of hypo-vigilance, the reminder will provide a stimulus to wake the officer's attention, e.g., an electrical pulse to wake an officer getting drowsy behind the wheel; or in the case of hyper-vigilance, a stimulus reminding the officer to engage in an action, e.g., take a deep breath.

Another potential outcome of this wearable technology is for law enforcement to actively monitor an officer's biometrics while in the field eyeing for extreme biometric variances. In this example, dispatchers, supervisors and/or others who are alerted to an unusual variance could directly intervene by checking on an officer's welfare or status. A spike in certain biometrics could indicate a health emergency or that an officer is physically struggling with an adversary. In either circumstance, support resources could be dispatched to the officer's location especially if voice communications have been compromised.

With the rationale firmly established, this prudent process will help prevent complex problems for law enforcement personnel where vigilant situational awareness is required.

Moreover, defined inputs, when properly analyzed, will deliver innumerable outputs that will transform and optimize a police officer's performance. As such, the shared datasets will assist police leaders, physicians, sleep therapists, nutritionists and others in providing guidance to keep officers healthy and in the proper mindset. Again, both advanced mental and physical conditioning will help avert costly mishaps, allowing officers to serve their communities more effectively and as importantly, allowing officers to increase their overall health and resiliency to both acute and chronic stress.

The present disclosure relates to the measurement, cataloging, analysis and forecasting of defined biometrics captured by wearable sensors to identify and reduce stress; to enhance decision-making capabilities in the midst of stressful conditions and to facilitate real-time feedback to police officers in order to optimize performance, preserve public safety and to reduce both short- and long-term health impacts. The captured data, stored in a secure cloud and analyzed against existing individualized baselines, will identify increases in stress and will alert authorized recipients of abnormal readings. This feedback loop will be used in both reality-based training environments and in the field to build resiliency to stress.

The present disclosure comprises the use of wearable sensors on police officers to capture defined biometrics, including Heart Rate (HR) and Heart Rate Variability (HRV) to measure stress during specifically designed, controlled and evaluated reality-based training environments to develop individualized baselines. The longitudinal data, stored in a secure cloud, will be analyzed to develop an index that coalesces an individual's personal demographics, their biometric measurements, the defined training activity and the observed performance. This index will be used to develop an improvement or optimization plan for each participant and is the basis for the feedback loop to indicate if real-time measurements are within, or outside of, the established normal margins.

Further, an alert is sent to the subject and/or the authorized recipients to warn of the abnormal readings. Using machine learning and artificial intelligence, the platform will provide predictive measures that forecast behaviors and/or actions; whereby, prescriptive remedies are available to the police officer to best manage the event. As such, the better-informed responder can adapt situational management to improve performance and provide for optimized outcomes.

### DESCRIPTION OF THE DRAWINGS

FIG. 1 an example platform as described herein.

FIG. 2 shows an example index including the subject's demographic data (e.g., age, gender, years of experience, biomarkers, etc.), longitudinal biometric readings (HR and HRV), and designed, controlled and evaluated reality-based training.

FIG. 3 shows an example monitoring loop that analyzes real-time data against the established baseline index to alert the subject and/or authorized recipients of their status, including providing suggested remedies when the readings are out-of-margin.

FIG. 4 shows an example optimization engine that uses the longitudinal indices to predict a subject's behavior; whereby, corrective actions are prescribed and performance is optimized during stress-induced events.

FIG. 5 shows an example method for creating and using a stress index.

### DETAILED DESCRIPTION

Through the use of wearable technology and the proper analysis of the data, emergency responder organizations will have the capacity to build both optimized performance and resiliency with its most important assets its personnel.

Preparedness, performance and prediction are the platform's goals:

Preparedness=Individual biometric baseline development using: Heart Rate (HR), and Heart Rate Variability (HRV) data in conjunction with biomarkers, demographics (age, gender, years of experience, etc.), and medical history data.

Performance=Real-time biometric data provide feedback to correct and influence performance (additionally, the capture of real-time data allows for longitudinal analysis to detect trends and understand the onset of chronic conditions).

Prediction=Baseline profiles balanced against real-time biometrics using the calculated index will prescribe corrective courses of action resulting in optimized performance and resiliency and will assist in the prediction of future behavior.

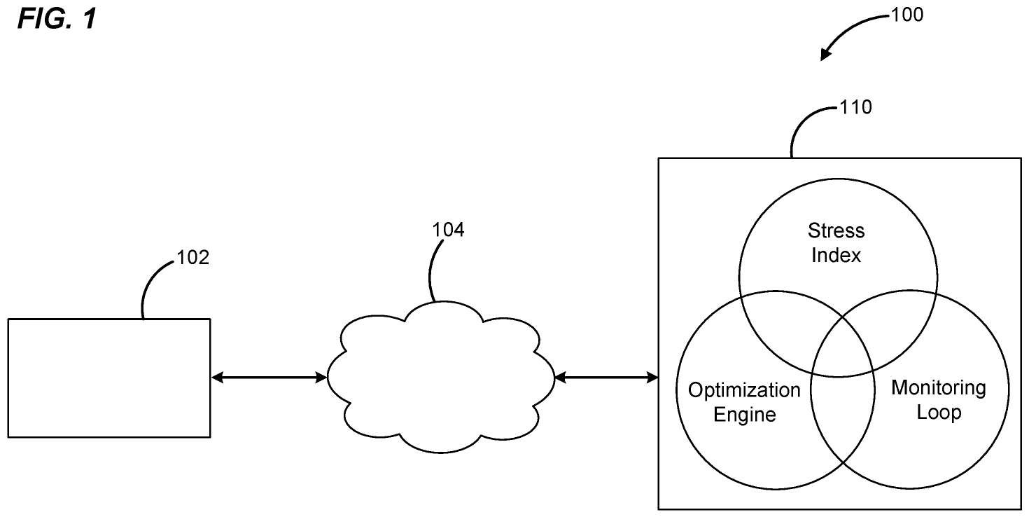

The platform 100, as shown in FIG. 1, is an interrelated suite of devices, software tools, analytics and specialty data stores bundled and delivered as a ‘Software as a Service’(SaaS). Its purpose is to assist those individuals in critical public service roles who desire to engage the Platform to understand the limits of their performance and to seek interventions to optimize their performance. Further, it is designed to offer benefit if only used once or if used throughout an individual's life of public safety service. This patent application seeks to lay claim to this platform being uniquely used to understand and mitigate the effect of acute and chronic stress in line of duty.

The platform's core is an objective measure of stress. There are many measures of stress comprising of heart rate monitoring, respiration, blood pressure, biomarkers, galvanic skin response, eye/pupil observations, and others that will can be incorporated into the platform.

The platform includes one or more devices 102 that measure the various physiological parameters of the individual, such as heart rate, respiration, etc. As noted, the device 102 is typically wearable technology that is worn on the individual, such as a public safety person. In one example, the device 102 is a wristwatch that measures various physiological attributes associated with the individual. Other or multiple devices can be used to gather the physiologic measurements.

The device 102 communicates over a network 104 to a server 110 of the platform 100. In some examples, the network 104 includes wired or wireless components and leverages various communications schemes, such as Wi-Fi, Bluetooth, cellular, etc. Other configurations are possible.

The server 110 is one or more devices (and associated databases), such as a server farm that leverages the computing capacity of many devices to receive, analyze, store, and act upon the data from the device 102, along with the data from thousands or millions of other similar devices associated with other individuals. The server 110 can be divided into logical groupings including a stress index, a monitoring loop, and an optimization engine, as described further below.

These components of the platform 100 are deployed in concert to understand a person's stress capacity, his or her resilience under pressure and to provide feedback and real-time tools to improve and optimize a person's performance under stress. The platform 100 is made up of four major components:

### Measuring and Indexing

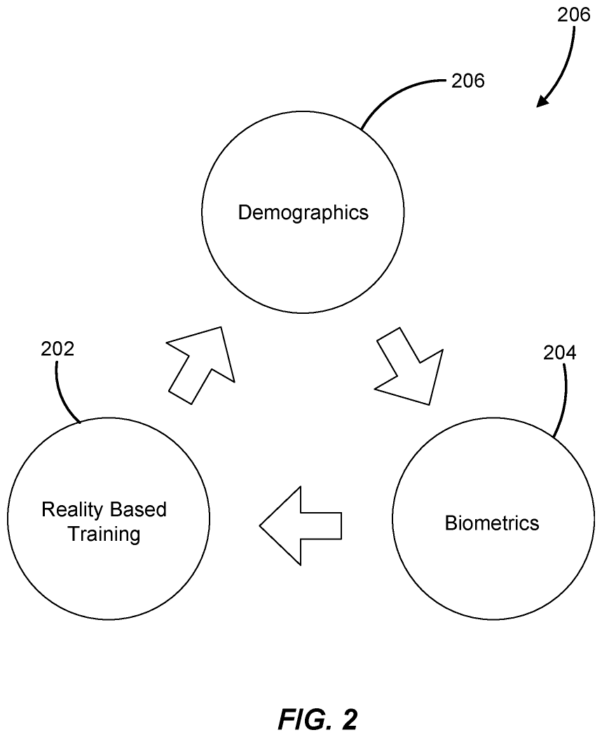

This component (see FIG. 2) involves the creation of a custom, personalized stress index 200: the index for individuals in a controlled testing environment using biometrics to measure stress and visual observation to measure performance. This component is generally the first completed it may be the only engagement, or the first of many interactions within the platform 100.

Baselining—The baseline stress index can be helpful in guiding those who seek to better their performance under stress. These measurements are made in a lab environment where the number of external, exogeneous factors can be reduced and/or controlled. A subject is outfitted with one or more biosensors. In the case of this index, the key sensors will capture: heart rate—EKG/ECG, respirations and acceleration. The individual will then be taken through a number of calibrated stress scenarios 202.

In each of these simulations, the person's biometric information 204 will be captured and stored. The calibrated scenarios will simulate stressful situations of varying magnitude. Examples include sleep, sedentary activity, active movements (exercise, general mobility), agitation, fight/flight responses, and overwhelming encounters. The simulations may be performed in different sequences and with more repetitions at each level. After the simulations, the data is consolidated, and an index is calculated for each stress level. These respective values then represent that person's baseline.

As the number of indexes calculated increases and is combined with real-world feedback, the ability to aggregate people into clusters with similar demographics, psychographics and biomarkers 206 will allow inferences to be made on future behavior, which will lead to better performance.

Longitudinal While the initial baseline can be helpful in assessing stress measures at a specific point in time, more powerful insight comes from measuring a person's stress index at periodic intervals and then analyzing the changes. By putting individuals through the same calibrated stress simulations at periodic intervals, a longitudinal profile will be determined giving these individuals and their counselors a mechanism to assess both gradual and abrupt changes in a person's stress indexes, which could show the effects of age, the tell-tale signs of stress e.g. PTSD, or the effect of life-changing events.

Monitoring biometric data offers a window into a person's life. Whether the data is cataloged on all aspects of a person's life, or just during times that are relevant, monitoring any changes to the data provides understanding on how the events of daily- and/or work-life contribute to a person's ability to manage stress. The findings will also illustrate the key factors required to improve one's ability to handle stress. The flow of data allows the monitoring process to become intelligent as data is directed to machine learning and other artificial intelligence algorithms that enables the identification of patterns in observable data, and builds models that explain and predict performance without having explicit pre-programmed rules and models.

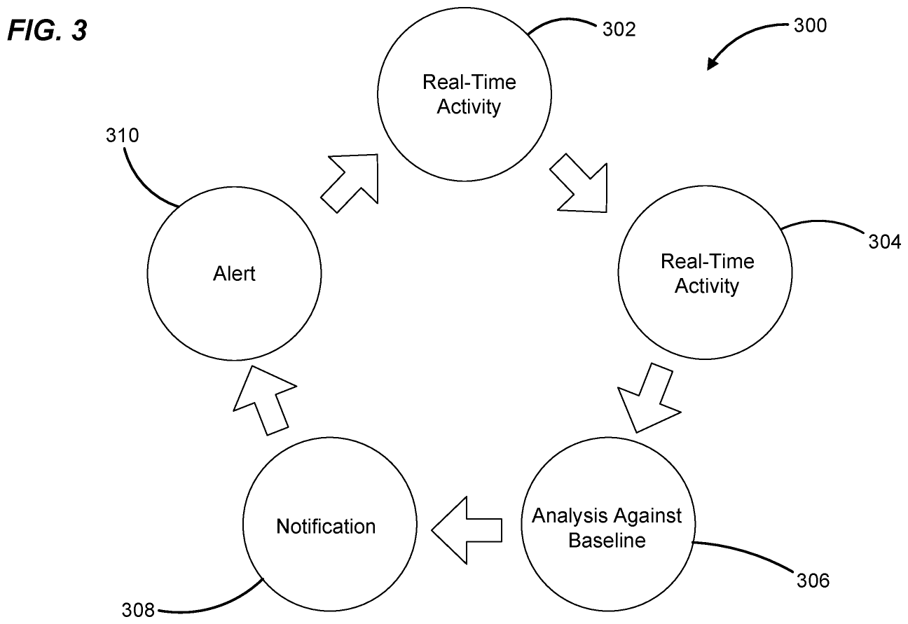

The use of intelligent biometric sensors offers law enforcement the unique opportunity to collect data from an individual continuously or periodically. The monitoring loop 300, a component of the platform 100 shown in FIG. 3, facilitates the capture of the biometric data 302, 304; the transmission of the data to the data store; the analysis 306 of the data (where machine learning and other artificial algorithms exist), where results are fed to the visualization engine and back to the user, providing feedback 308, 310; and offers prescriptive measures to assist with real-time or long-term performance optimization.

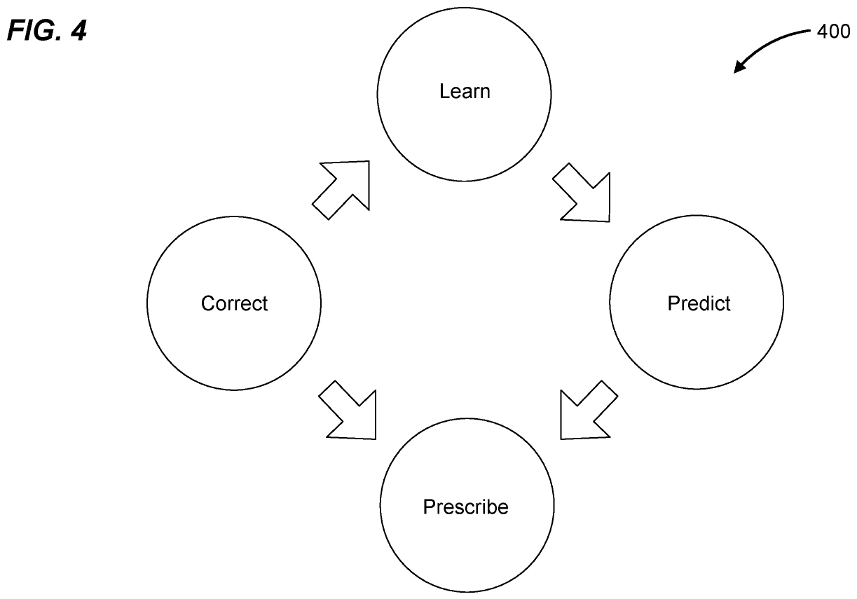

The incoming data stream 302, 304—from both the real-time monitoring and the controlled indexing and baselining—is directed through the optimization engine 400 (see FIG. 4) on its way to the data store. The engine 400, with access to past data, continuously analyzes the incoming data, detecting patterns and relationships and then extrapolating these relationships forward in time. The prescriptive portion of this process also monitors incoming data and via machine learning is ready with the best alternative or intervention to maximize the performance objective.

One of the end objectives of the platform is to provide direct feedback to an individual making them aware of insights from the optimization engine that would allow him or her to understand the end state of predicted performance and provide the best interventions (knowing the person's stress characteristics) to alter the course of events to achieve a better result. This portion of the platform initiates a direct connection back to the remote device, e.g., a cellphone running a mobile application, and push a stream of information that is interpreted by the app.

This information can either trigger an alert, provide haptics, text or text-to-speech that offers pertinent descriptive, predictive and prescriptive information that helps direct a person's actions. Additionally, the data stream back to the individual has a visualization component that provides app delivered tools that facilitate the person's ability to self-discover their current optimal stress performance state.

Simple reporting could be provided at any time for real-time or historical information. An alert system would send an immediate notice to the individual that their stress level is increasing at a level that is not consistent with their baseline. In addition, a notification could/would be sent to authorized recipients.

There are various ways to estimate the stress index 200. For one non-limiting example, beat-to-beat heart rate variability (HRV) is caused by a fluctuating balance between sympathetic tone and parasympathetic tone at the sinoatrial node. It is used for markers of autonomic modulation of the heart. The standard methods for analyzing HRV include: statistical (time domain), power spectral (frequency domain) and nonlinear geometrical analysis. Time and frequency domain methods provide a noninvasive, linear means of assessing the regulation of the autonomic system. The model proposed is based on a nonlinear approach.

The ease of HRV collection and measurement coupled with the fact it is relatively affordable, non-invasive and pain free makes it widely accessible. It is indeed a surrogate measure that can be easily deployed; can be captured with the current generation of biometric sensors; allows personalization for each individual; allows for longitudinally tracking monitor changes; and even though the measure may offer less information in an absolute sense, it will lend itself to further classification along psychographic parameters.

The Poincare plot provides a qualitatively and quantitatively visual measure of autonomic nervous system activity. The plot of the R-R intervals on a Poincare diagram with ellipse determined by SD1 and SD2 serves two purposes: 1) It provides each individual with a visual aid to adjust their autonomic balance toward improved levels for a current event via supervening interventions in real-time. Visually, this would be ‘sighting-in’ the current combination of real-time ellipse, SD1 and SD2 into the person's optimal ellipse, SD1 and SD2. 2) The graphic depiction also sets the stage for a showing the effects of machine learning and other artificial intelligence techniques on the iteration toward an adjusted optimal as new information is analytically digested by the platform.

The above formulae calculate the SD1 and SD2 directly from a specified data set. The formula for the ellipse on the Poincare plot with SD1 representing the major radius and SD2 the minor radius is plotted using the following relationship:
The Formula of a ROTATED Ellipse is:

$$\frac{{( ( X - {C}_{x} ) cos ( θ ) + ( Y - {C}_{y} ) sin ( θ ) )}^{2}}{{( {R}_{x} )}^{2}} + \frac{{( ( X - {C}_{x} ) sin ( θ ) - ( Y - {C}_{y} ) cos ( θ ) )}^{2}}{{( {R}_{y} )}^{2}} = 1$$

There:
(Cₓ, C_{y}) is the center of the Ellipse.
Rₓ is the Major-Radius, and R_{y} is the Minor-Radius.
θ is the angle of the Ellipse rotation.

In another non-limiting example, the stress index 200 is calculated by using beat-to-beat heart rate variability (HRV) that is caused by a fluctuating balance between sympathetic tone and parasympathetic tone at the sinoatrial node. It is used for markers of autonomic modulation of the heart. Methods for analyzing HRV include: statistical (time domain), power spectral (frequency domain) and nonlinear geometrical analysis. Time and frequency domain methods provide a noninvasive, linear means of assessing the regulation of the autonomic system.

A generalized expression of the stress index 200 is as follows:

S=f(PNS, SNS, BvSI, PoSI, Ia)

Where:
PNS=f(Mean RR, RMSSD, % SD2)
   Parasympathetic nervous system (PNS) activity (vagal stimulation) is known to decrease heart rate and increase heart rate variability
SNS=f(Mean RR, RMSSD, % SD1)
   Sympathetic nervous system (SNS) activity having the opposite effect on heart rate and heart rate variability, i.e. it increases HR and decreases HRV
BvSI=f(Amo, Mo, M×DnMn)=(AMo×100%)/(2×Mo×M×DMn)
   Baevsky's stress index (BvSI)—index of cardiovascular system stress and is strongly linked to sympathetic nervous activity
PoiSI==SD1/SD2
   Poincare nonlinear measures
Ia=Individual attributes (e.g. gender, age, height weight, experience, fitness level . . . )

As the stress index 200 is uniquely calculated to represent a particular individual's stress at a specific stress level:

[symbol]

(t)=(s₁s₂s₃ . . . siⱼ . . . sₘ)

Further, an individual's stress index at each stress level over time is represented as a two-dimensional matrix:

$${s}_{i} = [ {s}_{j , t} ] = [ \begin{matrix}{s}_{1 , 1} & … & {s}_{1 , m} \\ {s}_{1 , 2} & … & {s}_{2 , m} \\ {s}_{1 , 2} & … & {s}_{3 , m} \\ ⋮ & ⋮ & ⋮ \\ {s}_{1 , m} & … & {s}_{n , m}\end{matrix} ]$$

Where:
t=index of time
i=index of individual
j=index of calibrated stress (with j=1 representing least stress)
m=number of stress levels
n=number of time periods

As noted, the Poincare plot analysis is a geometrical and nonlinear method to assess the dynamics of HRV. It is a diagram in which each R-R interval (time between heartbeats) is plotted as a function of the previous R-R interval where the values of each pair of successive R-R intervals define a point in the plot. The Poincare plot is a geometrical representation that permits the visual identification of the presence of non-linear HRV components. The Poincare plot also provides a set of quantitative measures of the autonomic nervous system activity.

In the Poincare plots, the SD1 width reflects the parasympathetic activity; and the SD2 length reflects the sympathetic modulation. The determination of SD1 and SD2 follows:
Let RR, x and y vectors be defined as

RR≡(RR₁,RR₂, . . . ,RRₙ,RRₙ₊₁),

x≡(x₁,x₂, . . . ,x₃)≡(RR₁,RR₂ . . . RRₙ) (I)

y≡(y₁,y₂, . . . ,yₙ)≡(RR₂RR₃, . . . ,RRₙ₊₁),

where RR₁ stands for the ith RR interval, and n is the number of points in the Poincare plot (which is one less than the length of the RR time series). Using the above, the Poibcare plot of RR intervals can be defined as the ordered pairs (points in a plane) (xᵢ, yᵢ) (Piskorski and

$${d}_{i}^{1} = \frac{\vert ( {x}_{i} - {x}_{C} ) - ( {y}_{i} - {y}_{C} ) \vert}{\sqrt{2}} , \\ {d}_{i}^{2} = \frac{\vert ( {x}_{i} - {x}_{C} ) + ( {y}_{i} - {y}_{C} ) \vert}{\sqrt{2}} , \qquad ( 4 )$$

Consequently, we obtain the followin formulae for SD1_{C} and SD2_{C}:

$$\begin{matrix}SD {1}_{C}^{2} = \frac{1}{n} {\sum }_{i = 1}^{n} {( {d}_{i}^{1} )}^{2} , & ( 5 ) \\ SD {2}_{C}^{2} = \frac{1}{n} {\sum }_{i = 1}^{n} {( {d}_{i}^{2} )}^{2} . & ( 6 )\end{matrix}$$

Other calculated parameters that play an intermediate role in estimating the stress index 200 can include:

| Parameter | Units | Description |
|---|---|---|
| RR interval | | Time elapsed between two successive R-waves of the |
| | | QRS signal on the electrocardiogram |
| RR | [ms] | The mean of RR intervals |
| STD RR (SDNN) | [ms] | Standard deviation of RR intervals |
| HR | [beats/min] | The mean heart rate |
| Min & Max HR | [beats/min] | Minimum and maximum HR computed using N beat |
| | | moving average (default value: N = 5) |
| RMSSD | [ms] | Square root of the mean squared differences between |
| | | successive RR intervals |
| NNxx | [beats] | Number of successive RR interval pairs that differ more |
| | | than xx ms (default value xx: = 50) |
| pNNxx | [%] | NNxx divided by the total number of RR intervals |
| HRV triangular | — | The integral of the RR interval histogram divided by the |
| index | | height of the histogram |
| TINN | [ms] | Baseline width of the RR interval histogram |
| Spectrum | | Welch's (or Lomb-Scargle) periodogram and AR |
| | | spectrum estimates |
| Peak frequency | [Hz] | VLF, LF, and HF band peak frequencies |
| Absolute power | [ms2] | Absolute powers of VLF, LF, and HF bands |
| Absolute power | [log] | Natural logarithm transformed values of absolute |
| | | powers of VLF, LF, and HF bands |
| Relative power | [%] | Relative powers of VLF, LF, and HF bands: |
| | | VLF [%] = VLF [ms2]/total power [ms2] × 100% |
| | | LF [%] = LF [ms2]/total power [ms2] × 100% |
| | | HF [%] = HF [ms2]/total power [ms2] × 100% |
| Normalized power | [n.u.] | Powers of LF and HF bands in normalized units: |
| | | LF [n.u.] = LF [ms2]/(total power [ms2] − VLF [ms2]) × |
| | | 100% |
| | | HF [n.u.] = HF [ms2]/(total power [ms2] − VLF |
| | | [ms2]) × 100% |
| LF/HF | — | Ratio between LF and HF band powers |
| D2 | — | Correlation dimension |
| AMo | | the mode amplitude presented in percent or the height |
| | | of the normalized RR interval histogram |
| Mo | | the median of the RR intervals |
| MxDMn | | the difference between longest and shortest RR interval |
| | | values |

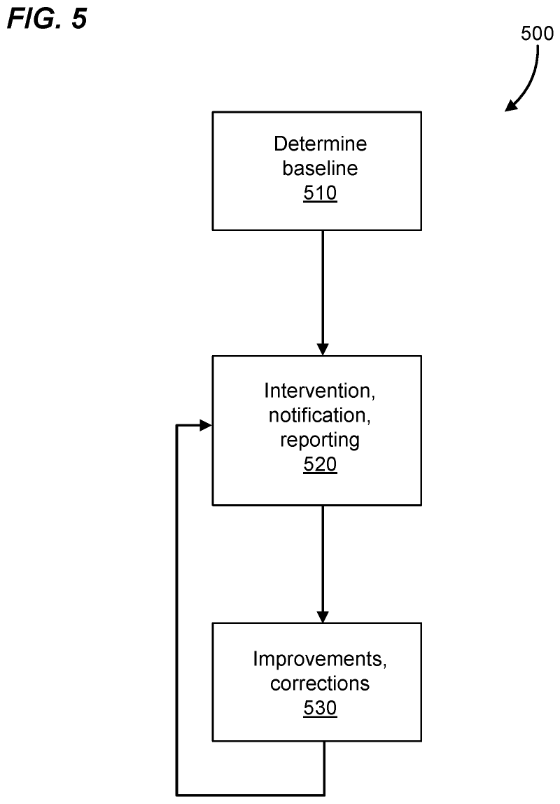

Referring now to FIG. 5, an example method 500 for creating and using a stress index is provided with the platforms described herein.

At operation 510, a baseline is determined. This baseline can be individualized, as described above. In operation 510, an emergency responder wears a device that measures biometrics including heart rate and heart rate variability. The responder is monitored during a set of activities including pre-determined training sessions and real life, on the job responses to incidents over a period of time. The specific time of each session or incident is recorded either by an observer or taken from the call dispatch records to determine the specific start and end time of each event.

When done in a training environment, performance is also evaluated to determine when the responder's performance is below set standards. The individual responder's biometrics and performance are converted into the index and the index is matched up with the specific event. When this is done over a series of event, a base line stress index is determined for this responder.

Next, at operation 520, interventions, notifications, and reporting are provided based upon the baseline stress index calculated for the emergency responder. Specifically, at operation 520, the devices worn by the responder allows for real-time monitoring by the responder or by others allowed to view the results and information. When a responder's biometrics are outside of the baseline, either below or above, a notification is given (e.g., a verbal command, a pulse provided by a device, a warning sound, etc.) indicating a range fault.

In addition, this information is recorded and monitored, which, in turn, is used to forecast or determine possible future faults or difficult events this responder should not handle. A reporting system matches up the specific events (training or on the job) and monitors the responder's stress index level to each of the events to determine if: 1) the baseline is changing over time; 2) if there is a range fault, it allows for incident review to determine if there was a significant event that caused the fault; and 3) with the use of AI, it is possible to predict the types of events that will cause a biometric fault thus increasing the risk of a poor outcome.

Next, at operation 530, improvements and corrective measures are performed to continually improve the stress index. This is an iterative process, with operations 520 and 530 being repeated. In addition, a responder's index, by activity, can be used to create tailored training programs, whereas trainers know what activities need to be a focus. In addition, the information may also be shared with the appropriate individuals to determine other concerns including a medical condition, a sleep disorder, or a mental health disorder.

There can be various technical advantages associated with the platforms described herein. For example, the platforms provide a more holistic approach to calculating a stress index for safety personnel. This results in a more efficient system that collects data from disparate sources, meaningfully analyzes that data, and can act on the results.

More specifically, through the efficient processes described herein, the stress index can be tailored for safety personnel to meet and exceed the challenges of their jobs. In real-time, biometrics can provide feedback to correct and influence performance. And, these measurements can help predict future behavior and prescribe corrective courses of action resulting in optimized performance and resiliency.

Data extracted from aperiodic and continuous biometric monitoring can be used to measure/understand stress and to and improve individual performance. A customized stress index can be determined for each person at each level of stress. For instance, a stress index can be constructed from heart rate variability (HRV) captured by wearable sensors. A baseline stress index can be determined, optimized for an individual to improve performance in real-time and used less frequently to understand changes in a safety person's baseline over time. The stress index provides an objective measure to customize training for each individual.

A stress index, in both absolute terms and relative changes to diagnose an individual's stress related conditions, can provide measures to direct prescriptive therapies—establishing the right mindset, wellness, fitness, behavioral health and specific counseling . . . all of which will different for each individual. Real-time presentation of the stress index can be used as a self-guide for the individual to better handle stress and control their phyco-physiological response. Further, the stress index can be used to predict performance in real-time.

Changes in the stress index can be used as an early indicator of chronic disorders/conditions like PTSD. Creating a baseline stress index and monitoring that stress index can and will play a major role in both understanding an individual's stress profile, managing that profile for changes over time and getting the individual the right help at the right time.

In some implementations, the principles described herein are used by an individual (e.g., emergency personnel) to: 1) establish a personal baseline; 2) discover the edges of the individual's baseline; and 3) learn different interventions/techniques to better endure stress, make better decisions, recover from a stressful incident and/or reduce the likelihood of chronic PTSD.

The example platforms described herein, including the devices used to collect the data, provide the monitoring loop and optimization engine, and calculate the personalized stress index, can be implemented as one or more computing devices. In these examples, the computing devices including at least one processor and non-transitory memory encoding instructions which, when executed by the at least one processor, cause the processor to implement the operations and other functionality described herein.

What is claimed is:

1. A platform, comprising:
at least one processor; and
memory encoding instructions which, when executed by the at least one processor, cause the at least one processor to:
receive biometric information from a plurality of public safety personnel;
analyze the biometric information to calculate a stress index; and
use the stress index to define and monitor stress in the public safety personnel.

2. The platform of claim 1, further comprising instructions which, when executed by the at least one processor, cause the at least one processor to use the stress index to monitor performance in public safety personnel.

3. The platform of claim 1, further comprising instructions which, when executed by the at least one processor, cause the at least one processor to use a vector of indexes to define stress at varying levels of cognitive engagement for the public safety personnel.

4. The platform of claim 1, further comprising instructions which, when executed by the at least one processor, cause the at least one processor to use a longitudinal series of stress indexes to facilitate therapeutic interventions.

5. The platform of claim 1, further comprising instructions which, when executed by the at least one processor, cause the at least one processor to use a Poincare plot and a series of estimated ellipses to provide visual tools to help balance stress, improve performance and optimize performance.

6. The platform of claim 1, further comprising one or more body worn sensors configured to capture the biometric information, including: Heart Rate, Heart Rate Variability, Respirations and Acceleration.

7. The platform of claim 1, further comprising instructions which, when executed by the at least one processor, cause the at least one processor to use Heart Rate Variability calculations captured during controlled reality-based training exercises to determine a personalized stress index.

8. The platform of claim 7, wherein the personalized stress index is used to establish a longitudinal baseline.

9. The platform of claim 1, further comprising instructions which, when executed by the at least one processor, cause the at least one processor to monitor in-the-field performance through the capture of defined biometrics.

10. The platform of claim 1, further comprising instructions which, when executed by the at least one processor, cause the at least one processor to report variations to a Poincare plot to visualize stress.

11. A method for calculating a stress index for a public safety person, the method comprising:
receiving biometric information from a plurality of public safety personnel;
analyzing the biometric information to calculate a stress index; and
use the stress index to define and monitor stress in the public safety person.

12. The method of claim 11, further comprising using the stress index to monitor performance in the public safety personnel.

13. The method of claim 11, further comprising using a vector of indexes to define stress at varying levels of cognitive engagement for the public safety personnel.

14. The method of claim 11, further comprising using a longitudinal series of stress indexes to facilitate therapeutic interventions.

15. The method of claim 11, further comprising using a Poincare plot and a series of estimated ellipses to provide visual tools to help balance stress, improve performance and optimize performance.

16. The method of claim 11, further comprising using one or more body worn sensors configured to capture the biometric information, including: Heart Rate, Heart Rate Variability, Respirations and Acceleration.

17. The method of claim 11, further comprising using Heart Rate Variability calculations captured during controlled reality-based training exercises to determine a personalized stress index.

18. The method of claim 17, further comprising using the personalized stress index to establish a longitudinal baseline.

19. The method of claim 11, further comprising monitoring in-the-field performance through the capture of defined biometrics.

20. The method of claim 11, further comprising:
establishing a personal baseline;
discovering boundaries of that baseline; and
providing different techniques to better endure stress.
